(19) United States
(12) Patent Application Publication
(10) Pub. No.: US 2010/0198292 A1
(43) Pub. Date: Aug. 5, 2010

(54) EVALUATING ELECTRODE CONFIGURATIONS FOR DELIVERING CARDIAC PACING THERAPY

(75) Inventors: Jordon D. Honeck, Maple Grove, MN (US); Robert T. Sawchuk, Roseville, MN (US)

Correspondence Address: Medtronic, Inc., 710 Medtronic Parkway, Minneapolis, MN, 55432, US

(73) Assignee: Medtronic, Inc., Minneapolis, MN (US)

(21) Appl. No.: 12/363,187

(22) Filed: Jan. 30, 2009

(51) Int. Cl. A61N 1/365; A61N 1/37

(52) U.S. Cl. 607/17; 607/27

(57) ABSTRACT

Described techniques include delivering cardiac pacing therapy from a medical device to a chamber of a heart via a first electrode configuration and determining that the delivery of cardiac pacing therapy via the first electrode configuration inadequately captures the chamber. In response to such a determination, the medical device delivers cardiac pacing therapy to the chamber of the heart via a plurality of additional electrode configurations. The techniques further comprise determining a capture characteristic for each of the additional electrode configurations based on the delivery of cardiac pacing therapy to the chamber of the heart via the plurality of other electrode configurations. A new electrode configuration for cardiac pacing may be selected based on the capture characteristics of the various electrode configurations.

### TECHNICAL FIELD

The disclosure relates to medical devices and, more particularly, to medical devices that deliver cardiac pacing.

### BACKGROUND

Cardiac pacing is delivered to patients to treat a wide variety of cardiac dysfunctions. Cardiac pacing is often delivered by an implantable medical device (IMD), which in some cases may also provide cardioversion or defibrillation, if needed. The IMD delivers such stimulation to the heart via electrodes located on one or more leads, which are typically intracardiac leads.

At times, a cardiac pacing pulse may fail to capture the myocardium. For example, the electrode of the lead may have shifted or become entirely dislodged from an implant site. Loss of capture is detrimental to the efficacy of cardiac pacing.

Various methods exist for detecting loss of capture. In some examples, a first pair of electrodes delivers a pacing pulse, and a second pair of electrodes detects an electrical signal indicative of capture. In other examples, a device detects a mechanical contraction of the heart at the target site.

### SUMMARY

In general, the disclosure relates to techniques for evaluating a plurality of other electrode configurations when cardiac pacing delivered via a current electrode configuration fails to capture the myocardium. In an example system comprising an implantable medical device (IMD) coupled to one or more implantable leads, a plurality of electrode configurations are available to deliver cardiac pacing to a target chamber of the heart. The electrode configurations include electrodes located on the leads, and may also include one or more electrodes located on a housing of IMD. An electrode configuration comprises the electrodes selected to deliver pacing therapy, as well as their polarity. An electrode configuration defines, and may be referred to as, a pacing vector.

In some examples, an IMD or other medical device delivers pacing therapy to the heart via particular electrode configuration in a manner that captures the heart to evoke a contraction. To maintain delivery of effective pacing therapy, the IMD monitors the heart in conjunction with the delivery of pacing therapy via a particular electrode configuration to determine if the pacing therapy is capturing the heart. If it is determined that delivering pacing therapy via a particular electrode configuration is not capturing the heart, or not capturing it with adequate regularity, the IMD tests a plurality of electrode configurations available to deliver the pacing therapy to the same chamber as the particular electrode configuration that was determined to inadequately capture the heart with pacing therapy.

For example, the IMD may individually deliver pacing therapy via each of the plurality of additional electrode configurations to determine a capture characteristic, e.g., a capture threshold value, for each respective electrode configuration. Each of the additional electrode configurations may then be evaluated with respect to one another based on the determined capture characteristics. One of the additional electrode configurations may then be selected to replace the existing electrode configuration based on the evaluation. In some examples, the additional electrode configurations are tested in a sequence, e.g., by cycling through the additional configurations. In some examples, back-up pacing is maintained in the current or primary electrode configuration during the testing of the additional electrode configurations.

In one example, the disclosure is directed to a method comprising delivering cardiac pacing therapy from a medical device to a chamber of a heart via a first electrode configuration; determining that the delivery of cardiac pacing therapy via the first electrode configuration inadequately captures the chamber; delivering cardiac pacing therapy to the chamber of the heart via a plurality of additional electrode configurations in response to the determination that the delivery of cardiac pacing therapy via the first electrode configuration inadequately captures the chamber; and determining a capture characteristic for each of the additional electrode configurations based on the delivery of cardiac pacing therapy to the chamber of the heart via a plurality of additional electrode configurations.

In another example, the disclosure is directed to a medical device system comprising a stimulation generator configured to deliver cardiac pacing therapy to a chamber of a heart via a first electrode configuration; and a processor configured to determine that the delivery of cardiac pacing therapy via the first electrode configuration inadequately captures the chamber, wherein the stimulation generator is configured to deliver cardiac pacing therapy to the chamber of the heart via a plurality of additional electrode configurations in response to the determination that the delivery of cardiac pacing therapy via the first electrode configuration inadequately captures the chamber, wherein the processor is configured to determine a capture characteristic for each of the additional electrode vector configurations based on the delivery of cardiac pacing therapy to the chamber of the heart via a plurality of additional electrode configurations.

In another example, the disclosure is directed to a medical device system comprising means for delivering cardiac pacing therapy from a medical device to a chamber of a heart via a first electrode configuration; means for determining that the delivery of cardiac pacing therapy via the first electrode configuration inadequately captures the chamber; means for delivering cardiac pacing therapy to the chamber of the heart via a plurality of additional electrode configurations in response to the determination that the delivery of cardiac pacing therapy via the first electrode configuration inadequately captures the chamber; and means for determining a capture characteristic for each of the additional electrode configurations based on the delivery of cardiac pacing therapy to the chamber of the heart via a plurality of other electrode configurations.

The details of one or more examples of the disclosure are set forth in the accompanying drawings and the description below. Other features, objects, and advantages of the disclosure will be apparent from the description and drawings, and from the claims.

### BRIEF DESCRIPTION OF DRAWINGS

FIG. 1 is a conceptual diagram illustrating an example therapy system that provides cardiac pacing therapy to a patient.

FIG. 2 is a conceptual diagram illustrating a portion of the example therapy system of FIG. 1 in greater detail.

FIG. 3 is conceptual diagram illustrating a portion of another example therapy system similar to the system of FIG. 2.

FIG. 4 is a conceptual diagram illustrating another example cardiac therapy system.

FIG. 5 is a functional block diagram illustrating various components of an example implantable medical device.

FIG. 6 is a functional block diagram illustrating various components of another example implantable medical device similar to the device of FIG. 5.

FIG. 7 is a block diagram illustrating various components of an example programmer for programming an implantable medical device.

FIG. 8 is a schematic diagram illustrating various example pacing vectors available to an implantable medical device for delivery of pacing therapy to the heart.

FIG. 9 is a flow diagram illustrating an example technique for evaluating alternate pacing vectors.

### DETAILED DESCRIPTION

In general, the disclosure describes systems, devices and techniques relating to the delivery cardiac pacing therapy to the heart of a patient. In some examples, a cardiac pacing therapy system includes a medical device, such as an implantable medical device (IMD) that generates and delivers cardiac pacing therapy to the heart of a patient in the form of one or more electrical stimulation signals via one or more electrodes of an implantable lead connected to the IMD. Such a therapy system are configured to generate and deliver pacing therapy according to a variety of electrical stimulation parameters, which may include amplitude, pulse width, pulse rate, electrode combination, electrode polarity, and the like. The specific values for these parameters influence the effectiveness of the pacing therapy in achieving adequate capture the heart. Capture generally refers to the pacing therapy causing sufficient depolarization of the myocardium that a propagating wave of excitation and contraction results, which may be considered a heartbeat.

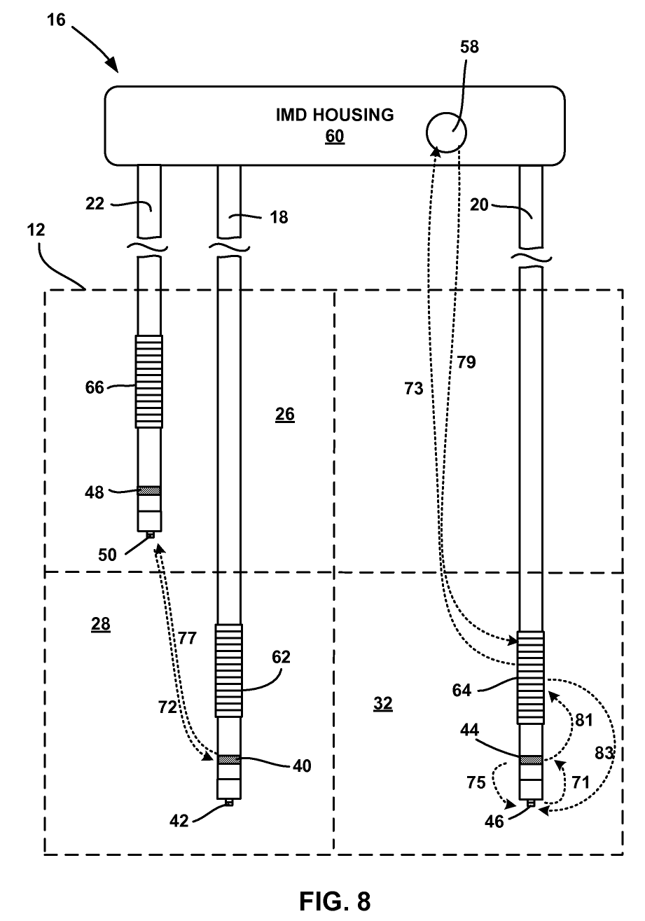

The electrode configuration with which cardiac pacing therapy system delivers cardiac pacing therapy may be referred to as a pacing vector. In most cases, a pacing vector refers not only the one or more electrodes on an implantable lead that deliver the electrical pacing signal but also the polarity of the electrodes. For example, a pacing vector configured for bipolar pacing stimulation may be defined by an anodic ring electrode on an implantable lead and a cathodic tip electrode on the same implantable lead. This and other examples of pacing vectors are described below with respect to FIG. 8. In any case, different pacing vectors may include not only different electrode combinations from one another, but also may include the same electrode combinations with different polarities.

Depending on the number of electrodes that a cardiac pacing therapy system includes, the cardiac pacing therapy system may be capable of delivering pacing therapy via a wide variety of pacing vectors to the same chamber of the heart of a patient. However, depending in part on the parameters of the electrical signal delivered via a pacing vector, such as pulse width, amplitude, and pulse rate, each one of these different pacing vectors may provide a different level of effectiveness with respect to the capture of the chamber of the heart. Furthermore, each of the vectors may have a different susceptibility to side effects, such as unintentionally stimulating the phrenic nerve.

Additionally, the effectiveness of a pacing vector may change over time for a wide variety of reasons. For example, dislodgment or micro dislodgement of one or more electrodes on an implantable lead configured to deliver pacing therapy may decrease the effectiveness of delivery of pacing therapy via a specific pacing vector. While a particular pacing vector may initially be found to provide adequate capture in the chamber of the heart at a certain time, e.g., during a programming session, the effectiveness of the delivery of pacing therapy via the pacing vector may change over time. Accordingly, in some cases, a pacing vector that initially provides adequate capture in the chamber of the heart of a patient may not provide adequate capture in the future.

The techniques described in this disclosure allow a therapy system to modify the delivery of cardiac pacing therapy to a chamber of the heart of a patient when it is determined that a specific pacing vector inadequately captures pacing pulses via one or more pacing vectors. For example, as will described in greater detail below, the cardiac pacing therapy systems described in this disclosure may determine that delivery of cardiac pacing therapy to a chamber of the heart of patient via a particular pacing vector is inadequately capturing the chamber of the heart, e.g., by failing to capture the chamber of the heart entirely or achieving capture of the chamber less than an acceptable percentage of times the therapy is delivered. When it is determined that delivery of pacing therapy to the chamber of is inadequately capturing the chamber, the cardiac pacing system responds by delivering cardiac pacing therapy to the same chamber of the heart via a two or more additional pacing vectors. For each of the additional pacing vectors, the cardiac pacing system determines a capture characteristic during the delivery of the cardiac pacing therapy. These capture characteristics may allow each of the pacing vectors to be evaluated with respect to one another. In this manner, a new pacing vector may be optimally selected from the additional pacing vectors for delivery of pacing therapy to replace the pacing vector that was determined to achieve inadequate capture within the chamber of the heart.

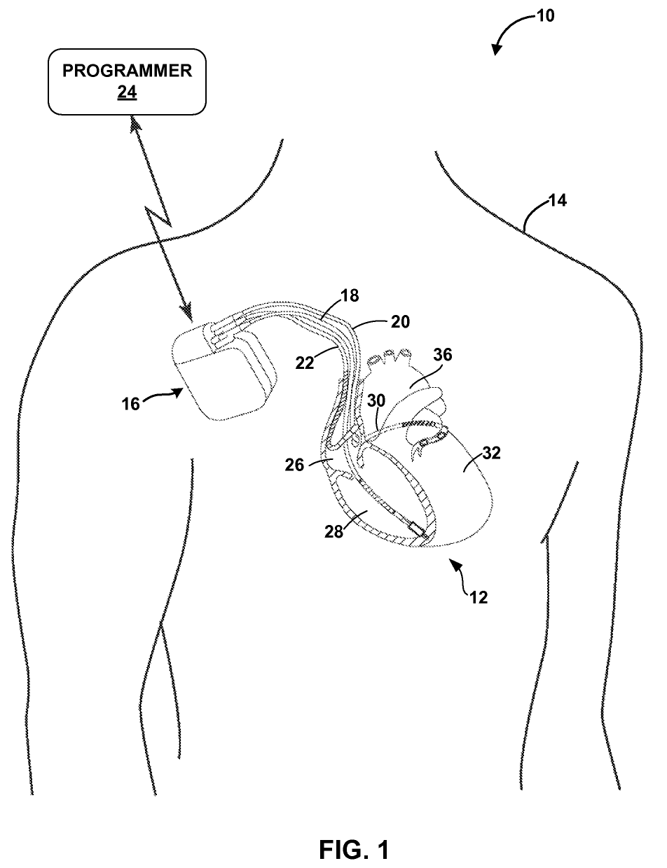

FIG. 1 is a conceptual diagram illustrating an example therapy system 10 that provides cardiac therapy to heart 12 of patient 14. Therapy system 10 includes an IMD 16, which is coupled to leads 18, 20, and 22, and a programmer 24. In the example of FIG. 1, IMD 16 comprises an implantable pacemaker, cardioverter, and/or defibrillator that provides electrical stimulation signals to heart 12 via electrodes coupled to one or more of leads 18, 20, and 22.

Leads 18, 20, 22 extend into the heart 12 of patient 16 to deliver electrical stimulation to heart 12 and/or sense electrical activity of heart 12. In the example shown in FIG. 1, right ventricular (RV) lead 18 extends through one or more veins (not shown), the superior vena cava (not shown), and right atrium 26, and into right ventricle 28. Left ventricular (LV) coronary sinus lead 20 extends through one or more veins, the vena cava, right atrium 26, and into the coronary sinus 30 to a region adjacent to the free wall of left ventricle 32 of heart 12. Right atrial (RA) lead 22 extends through one or more veins and the vena cava, and into the right atrium 26 of heart 12.

System 10 provides pacing therapy to heart 12 of patient 12 to manage the cardiac rhythm of heart 12. In particular, IMD 16 delivers pacing therapy in the form of electrical stimulation to heart 12 via a particular electrode configuration, e.g., a particular pacing vector. The delivery of electrical stimulation from IMD 16 via the pacing vector is intended to achieve capture i.e., sufficient depolarization of the myocardium of heart 12 so that a contraction results, of one or more chambers of heart 12. In some cases, IMD 16 also senses electrical signals within one or more chambers of heart 12 to monitor the intrinsic and/or evoked cardiac signals via one or more electrodes on leads 18, 20, or 22, e.g., to sense the need for delivery of pacing stimulation and/or determine proper timing for delivery of the electrical stimulation.

In some situations, pacing therapy delivered from IMD 16 to heart 12 via a particular pacing vector may inadequately capture heart 12. In such cases, system 10, e.g., IMD 16, determines that delivery of pacing therapy to a chamber of heart 12 via a particular pacing vector is inadequately capturing heart 12, e.g., by sensing for the cardiac signal evoked by the pacing therapy via one or more electrodes on leads 18, 20, and/or 22. Based on the determination of inadequate capture, IMD 16 may cycle through a plurality of additional pacing vectors capable of delivering pacing therapy to the same chamber of heart 12 to determine a capture characteristic for each additional pacing vector.

For example, IMD 16 may sequentially deliver pacing therapy to the chamber of heart 12 via each additional vector, and then determine a capture characteristic for each respective additional pacing vector based on delivery of the pacing therapy via the respective additional pacing vector. The capture characteristic determined for each respective additional pacing vector may be useful in evaluating the pacing vectors relative to one another in terms of the delivery of pacing therapy. In view of the capture characteristics determined for each additional pacing vector, IMD 16 may be reconfigured to deliver pacing therapy via one of the additional pacing vectors instead of the particular pacing vector that was determined to inadequately capture the chamber of the heart.

In some examples, external programmer 24 comprises a handheld computing device, a computer workstation, a home monitor device, or another other computing device. Programmer 24 may include a user interface that receives input from and/or presents information to a user. A user, such as a physician, technician, clinician, or other caregiver, interacts with programmer 24 to communicate with IMD 16.

The user may interact with programmer 24 to retrieve physiological or diagnostic information from IMD 16. As an example, the user may interact with programmer 24 to retrieve data relating to the inadequate capture detected with respect to the pacing therapy delivered via a first electrode configuration, and/or retrieve capture characteristic information determined for one or more additional electrode configurations in response to detection of inadequate capture with the primary configuration. A user may also interact with programmer 24 to program IMD 16, e.g., select values for operational parameters of the IMD. As an example, the user may interact with programmer 24 to select an alternate electrode configuration based on the capture characteristics of the electrode configurations.

In some examples, IMD 16 generates an indication that delivery of pacing stimulation via a particular pacing vector is not producing adequate capture. IMD 16 stores such an indication and/or transmits the indication by wireless telemetry to programmer 24 or another external device. Additionally, or alternatively, IMD 16 may generate an audible or tactile alert for the patient in the event that inadequate capture is detected. In response, patient 12 may elect to promptly visit the clinic for further evaluation of potential pacing condition that may alter reduce the effectiveness of the pacing therapy delivered by IMD 16 via a pacing vector.

In some examples, as will be described further below, upon identification of inadequate capture of a chamber of the heart with delivery of pacing therapy via a current electrode configuration, i.e., pacing vector, IMD 16 may reconfigure the pacing such that the pacing therapy is delivered to a different pacing vector. The new pacing vector is selected from a plurality of additional pacing vectors. To determine the new pacing vector, IMD 16 delivers pacing therapy to heart 12 of patient 12 via each additional pacing vector and determines a capture characteristic for each pacing vector during delivery of the pacing therapy. IMD 16 may then automatically select the new pacing vector may be selected based on the capture characteristics determined for each pacing vector relative to one another.

In other implementations, IMD 16 stores the capture characteristics of the additional pacing vectors, and analysis of such information is performed by programmer 24 or another external device. In this case, programmer 24 may retrieve information from IMD 16 for purposes of archival, processing and analysis in order to evaluate the capture characteristics of the additional pacing vectors.

In some cases, the capture characteristics relating to the additional pacing vectors obtained from IMD 16 is displayed to a user via a user interface of programmer 24 or any other suitable device for displaying such data to a user. A user may analyze the retrieved information, e.g., by visual inspection, and evaluate the capture characteristics of one or more the additional pacing vectors. In turn, based on the evaluation of capture characteristics, the user may indicate the new pacing vector for IMD 16 to deliver pacing therapy to heart 12 of patient 12, to ensure that the pacing therapy evokes adequate capture of the chamber of heart 12.

IMD 16 and programmer 24 may communicate with one another via wireless telemetry using any techniques known in the art. Examples of communication techniques may include, for example, low frequency or radiofrequency (RF) telemetry, but other techniques are also contemplated. In some examples, programmer 24 may include a programming head that may be placed proximate to the patient's body near the IMD 16 implant site in order to improve the quality or security of communication between IMD 16 and programmer 24.

In some examples, programmer 24 or another external device may be configured to allow remote programming of IMD 16 and/or the remote retrieval of stored data. For example, programmer 24 may include a home monitor device connected to an off-site network device which may communicate with the home monitor device to program IMD 16 and/or retrieve data stored on IMD 16. In this manner, one or more aspects of the disclosure may be performed by a device or user at a location that is remote from the patient.

Further, as a home monitor or handheld device, programmer 24 may be configured to provide one or more types of an alert to an off-site network device to communicate alerts to a user such as a clinician as a function of the detection of inadequate capture, and allow a user to properly and timely address any inadequate capture condition associated with the pacing therapy delivered by IMD 16. For example, a remote user may be alerted to the capture condition and provided with one or more capture characteristics determined for each pacing vector of a plurality of additional pacing vectors tested in response to the determined capture issue. Based on the capture characteristics provided for each respective pacing vector, the remote user may select a new pacing vector for the pacing therapy being delivered to patient 12.

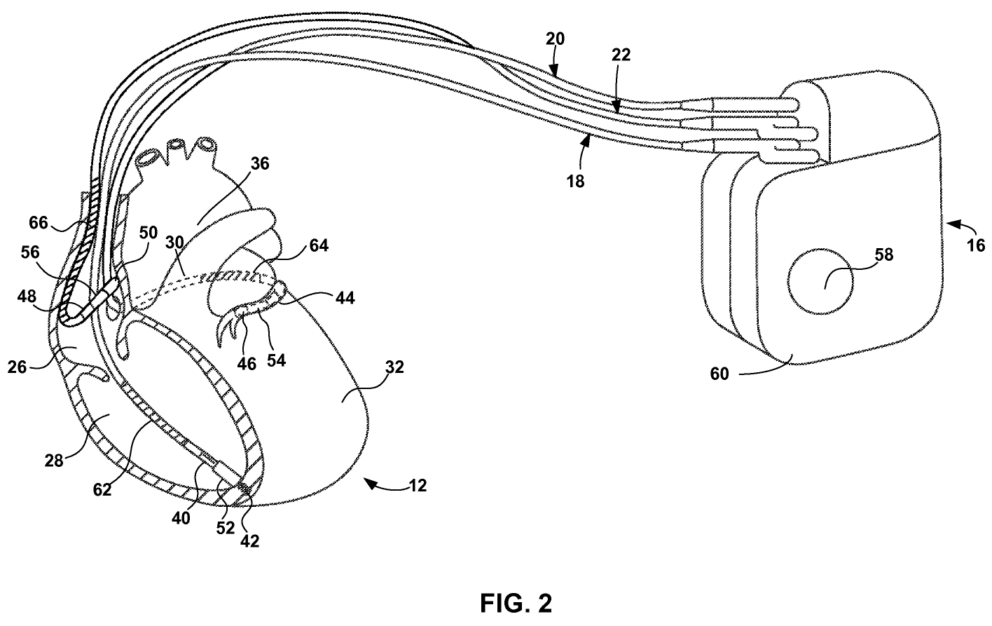

FIG. 2 is a conceptual diagram illustrating IMD 16 and leads 18, 20, and 22 of therapy system 10 in greater detail. Each of the leads 18, 20, 22 includes an elongated insulative lead body carrying a number of conductors. Bipolar electrodes 40 and 42 are located adjacent to a distal end of lead 18. In addition, bipolar electrodes 44 and 46 are located adjacent to a distal end of lead 20 and bipolar electrodes 48 and 50 are located adjacent to a distal end of lead 22. Electrodes 40, 44 and 48 may take the form of ring electrodes, and electrodes 42, 46 and 50 may take the form of extendable helix tip electrodes mounted retractably within insulative electrode heads 52, 54 and 56, respectively. Leads 18, 20, 22 may include elongated electrodes 62, 64, 66, respectively, which may take the form of a coil. Each of the electrodes 40, 42, 44, 46, 48, 50, 62, 64 and 66 may be electrically coupled to a respective one of the conductors within the lead body of its associated lead 18, 20, 22, and thereby electrically coupled to an implantable signal generator and an implantable sensing module within housing 60 of IMD 16.

In some examples, as illustrated in FIG. 2, IMD 16 includes one or more housing electrodes, such as housing electrode 58, which may be formed integrally with an outer surface of hermetically-sealed housing 60 of IMD 16 or otherwise coupled to housing 60. In some examples, housing electrode 58 is defined by an uninsulated portion of an outward facing portion of housing 60 of IMD 16. Other divisions between insulated and uninsulated portions of housing 60 may be employed to define two or more housing electrodes. In some examples, housing electrode 58 comprises substantially all of housing 60.

IMD 16 delivers pacing pulses via selected configurations of electrodes 40, 42, 44, 46, 48, 50, 58, 62, 64 and 66 to cause depolarization of cardiac tissue of heart 12. Selected configurations of electrodes 40, 42, 44, 46, 48, 50, 58, 62, 64 and 66 sense electrical signals attendant to the depolarization and repolarization of heart 12. These sensed signals may include those evoked by the delivering of pacing therapy to heart 12 of patient 14 via a particular pacing vector. These sensed cardiac signals may be used to determine whether the pacing therapy delivered via a particular pacing vector adequately captures heart 12, and also may be used to determine capture characteristic for each additional pacing vectors, as described herein. The electrical signals are conducted to IMD 16 via the respective leads 18, 20, 22. IMD 16 may deliver cardioversion or defibrillation shocks to heart 12 via any combination of elongated, coil electrodes 62, 64, 66, and housing electrode 58.

The configuration of therapy system 10 illustrated in FIGS. 1 and 2 is merely one example. In other examples, a therapy system may include epicardial leads and/or patch electrodes instead of or in addition to the transvenous leads 18, 20, 22 illustrated in FIG. 1. In addition, in some cases, IMD 16 may include one or more subcutaneous electrodes for sensing and delivery of pacing pulses and/or cardioversion-defibrillation energy. Further, a medical device capable of performing in accordance with this disclosure need not be implanted within patient 14. In examples in which a medical device not implanted in patient 14, the medical device may deliver pacing pulses and other therapies to heart 12 via percutaneous leads that extend through the skin of patient 14 to a variety of positions within or outside of heart 12.

Further, in some examples, an IMD includes or is coupled to one or more sensors configured to sense signals associated with one or more physiological parameters of a patient. Such sensors sense signals in addition to the signals sensed via one or more of electrodes 40, 42, 44, 46, 48, 50, 58, 62, 64, and 66 on leads 18, 20, and 22. In some examples, such sensors are used by an IMD to detect capture of heart 12 and/or determine capture characteristics of various electrode configurations.

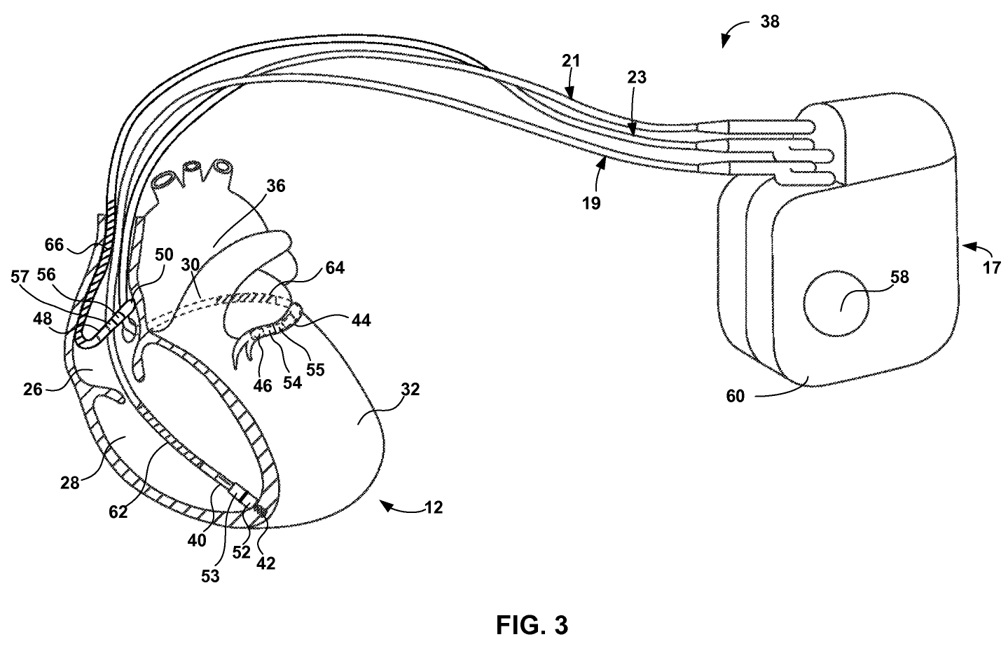

FIG. 3 is a conceptual diagram illustrating a portion of another example therapy system 38 similar to the portion of therapy system 10 illustrated in FIG. 2, except that leads, 19, 21 and 23 coupled to an IMD 17 further include lead-based, sensors 53, 55, and 57, respectively. In the example illustrated by FIG. 3, sensors 53, 55, and 57 are located adjacent to the distal end of leads 19, 21, and 23, between electrodes 40, 44 and 48, respectively. However, the location of sensors 53, 55, and 57 on leads 19, 21, and 23 is not limited to that illustrated in FIG. 3, but instead may be located at any suitable location along leads 19, 21, and 23, or other leads coupled to IMD 17. In some examples, one or more sensors are wirelessly coupled to IMD 17, or located within housing 60 of IMD 17. The number and locations of sensors 53, 55 and 57 illustrated by FIG. 3 is merely one example.

Sensors 53, 55, and 57 may comprise, as examples, oxygen sensors, accelerometers, or pressure sensors. In this manner, signals associated with one or more parameters, e.g., oxygen concentration, tissue perfusion, activity, posture, motion, blood pressure, or the like, may be sensed by IMD 17 in addition to the signals sensed by one or more of electrodes 40, 42, 44, 46, 48, 50, 62, 64, and 66 on leads 19, 21, and 23.

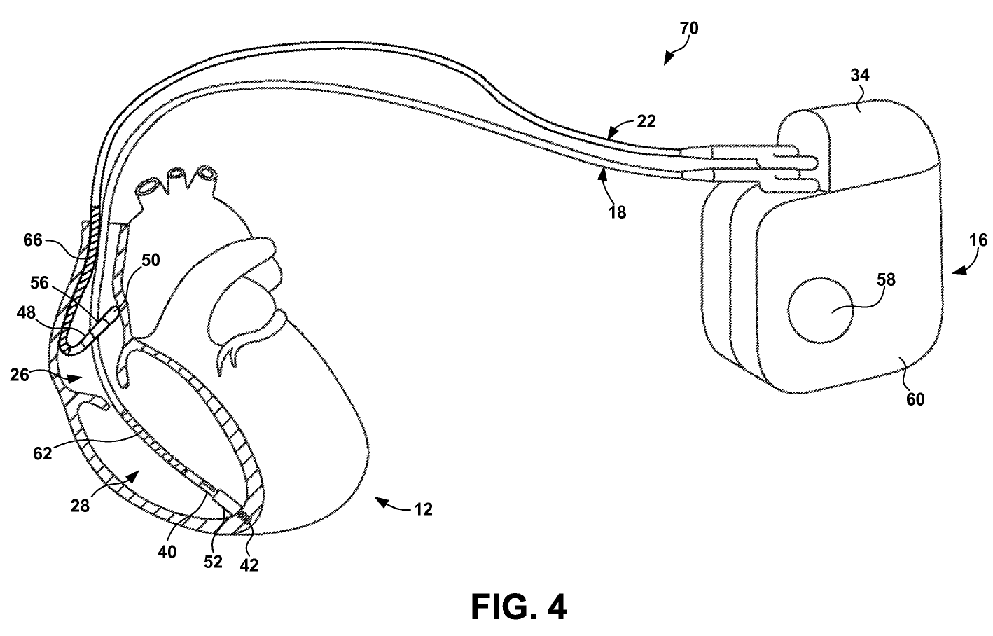

FIG. 4 is a conceptual diagram illustrating another therapy system 70, which is similar to therapy system 10 of FIGS. 1 and 2, but includes two leads 18, 22, rather than three leads. Leads 18, 22 are implanted within right ventricle 28 and right atrium 26, respectively. Therapy system 70 shown in FIG. 4 may be useful for providing pacing pulses and cardioversion-defibrillation shocks to heart 12, and for providing any of functionality described in this disclosure with respect to the systems of FIGS. 1-3.

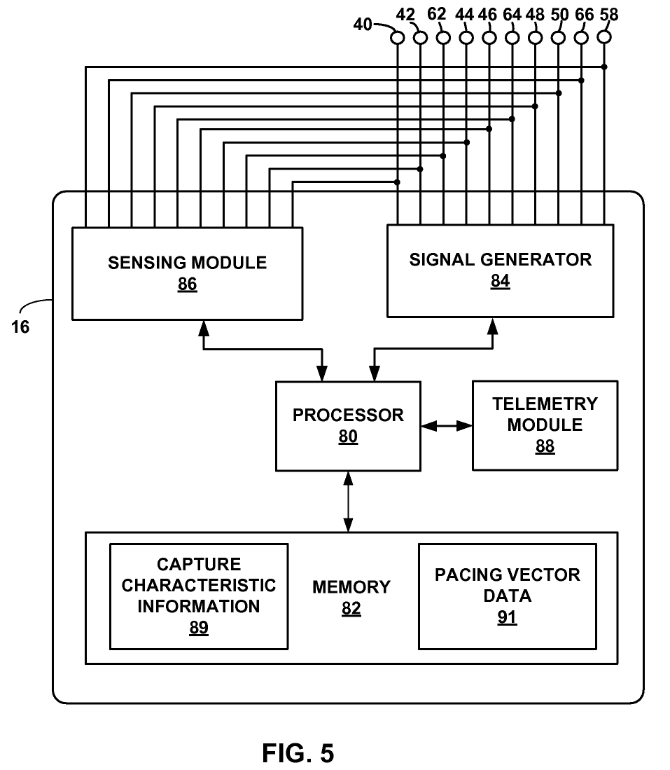

FIG. 5 is a functional block diagram of one example configuration of IMD 16. In the example shown in FIG. 5, IMD 16 includes a processor 80, memory 82, stimulation generator 84, sensing module 86, and telemetry module 88. Memory 82 includes computer-readable instructions that, when executed by processor 80, cause IMD 16 and processor 80 to perform various functions attributed to IMD 16 and processor 80 in this disclosure. Memory 82 also stores capture characteristic information 89 and pacing vector data 91, which are discussed in greater detail below. Memory 82 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media. Memory 82 may be a single memory module, or a combination of multiple memory modules including combinations of one or more types of memory as described above.

Pacing vector data 91 comprises data relating to some or all of the pacing vectors available for delivering pacing therapy to heart 12 of patient 12. Examples of possible pacing vectors of system 10 are described further in FIG. 8. In some examples, for each vector, pacing vector data 91 is sorted by or identifies the chamber of heart 12 to which pacing therapy is delivered via the pacing vector.

Processor 80 accesses pacing vector data 91 when it is determined that delivery of pacing therapy via a particular pacing vector is inadequately capturing the desired chamber of heart 12. In particular, processor 80 accesses pacing vector data 91 to determine the plurality of additional pacing vectors that are available for delivering pacing therapy to the same chamber of heart 12 other than that of the particular pacing vector that processor 82 has determined to inadequately capture the chamber of heart 12. Processor 80 may then control signal generator 84 to sequentially deliver pacing therapy to the chamber of heart 12 via one of the additional available pacing vectors until all of the additional pacing vectors are tested. Based on the effectiveness of pacing therapy via each of the additional pacing vectors, processor 80 may determine a capture characteristic for each respective additional pacing vector.

Capture characteristic information 89 comprises data relating to the capture characteristics determined for each pacing vector. In some examples, capture characteristic information 89 comprises capture threshold values determined for each respective additional pacing vector. Additionally or alternatively, capture characteristic information 89 for a pacing vector may simply indicate whether a respective additional pacing vector adequately captured the chamber of heart 12 when pacing therapy was delivered to the chamber via the pacing vector. Processor 80 may access the information stored in capture characteristic information 89 portion of memory 82 to facilitate the selection of a particular new pacing vector for delivery of pacing therapy to the chamber of heart 12 from among the additional pacing vectors that were tested.

Processor 80 may include one or more of a microprocessor, a controller, a digital signal processor (DSP), an application specific integrated circuit (ASIC), a field-programmable gate array (FPGA), or equivalent discrete or integrated logic circuitry. In some examples, processor 80 may include multiple components, such as any combination of one or more microprocessors, one or more controllers, one or more DSPs, one or more ASICs, or one or more FPGAs, as well as other discrete or integrated logic circuitry. Accordingly, processor 80 may refer to a single processing and control unit, or a combination of processing and control units, in whatever form or combination, useful in controlling the functionality of IMD 16. The functions attributed to processor 80 in this disclosure may be realized by software, firmware, hardware or any combination thereof.

Implantable signal generator 84 is configured to deliver cardiac pacing stimulation to cardiac tissue. Processor 80 controls signal generator 84 to deliver stimulation therapy to heart 12 according to a selected one or more of therapy programs, which may be stored in memory 82. Specifically, processor 80 may control signal generator 84 to deliver electrical pulses with amplitudes, pulse widths, frequency, and electrode configurations specified by the selected therapy programs. As discussed above, an electrode configuration comprises a combination of two or more electrodes selected for delivery of pacing therapy and the polarities of the electrodes, and may also be referred to as a pacing vector.

As shown in FIG. 5, signal generator 84 is electrically coupled to electrodes 40, 42, 44, 46, 48, 50, 58, 62, 64, and 66, e.g., via conductors of the respective lead 18, 20, 22, or, in the case of housing electrode 58, via an electrical conductor disposed within housing 60 of IMD 16. In some examples, signal generator 84 delivers pacing, cardioversion, or defibrillation stimulation in the form of electrical pulses or shocks. In other examples, signal generator 84 delivers one or more of these types of stimulation in the form of other signals, such as sine waves, square waves, or other substantially continuous time signals.

Signal generator 84 may include a switch module and processor 80 may use the switch module to select, e.g., via a data/address bus, electrodes to be used to deliver cardioversion-defibrillation shocks or pacing pulses. The switch module may include a switch array, switch matrix, multiplexer, or any other type of switching device suitable for selectively coupling stimulation energy to selected electrodes. In this manner, processor 80 may select through which electrode configuration, i.e., pacing vector signal generator 84 delivers pacing stimulation.

Sensing module 86 is configured to monitor one or more cardiac signals via selected combinations of electrodes 40, 42, 44, 46, 48, 50, 58, 62, 64 or 66, i.e., selected sensing vectors, in order to monitor electrical activity of heart 12. Sensing module 86 may also include a switch module by which processor 80 selects which pairs or combinations of electrodes are used to sense the heart activity. In some examples, sensing module 86 includes one or more sensing channels, each of which may comprise an amplifier. In response to the signals from processor 80, the switch module within sensing module 86 may couple selected electrodes to one of the sensing channels.

One channel of sensing module 86 may include an R-wave amplifier that receives signals from electrodes 40 and 42, which are used for pacing and sensing in right ventricle 28 of heart 12. Another channel may include another R-wave amplifier that receives signals from electrodes 44 and 46, which are used for pacing and sensing proximate to left ventricle 32 of heart 12. In some examples, the R-wave amplifiers may include an automatic gain controlled (AGC) amplifier that provides an adjustable sensing threshold as a function of the R-wave amplitude of the heart rhythm.

In addition, one channel of sensing module 86 may include a P-wave amplifier that receives signals from electrodes 48 and 50, which are used for pacing and sensing in right atrium 26 of heart 12. In some examples, the P-wave amplifier may include an automatic gain controlled amplifier that provides an adjustable sensing threshold as a function of the measured P-wave amplitude of the heart rhythm.

Examples of R-wave and P-wave amplifiers are described in U.S. Pat. No. 5,117,824 to Keimel et al., which issued on Jun. 2, 1992 and is entitled, “APPARATUS FOR MONITORING ELECTRICAL PHYSIOLOGIC SIGNALS,” and is incorporated herein by reference in its entirety. In general, R-wave and P-wave amplifiers are configured to output a signal to processor 80 when an R-wave or P-wave occurs in the relevant chamber. In some examples, one or more of the sensing channels of sensing module 84 may be selectively coupled to housing electrode 58, or elongated electrodes 62, 64, or 66, with or instead of one or more of electrodes 40, 42, 44, 46, 48 or 50, e.g., for unipolar sensing of R-waves or P-waves in any of chambers 26, 28, or 32 of heart 12.

In addition to detecting R-waves and P-waves, e.g., for controlling the timing and other aspects of the delivery of cardiac pacing, electrical signals from heart 12 monitored by sensing module 86 may be used to determine whether a pacing therapy delivered via a particular pacing vector achieves adequate capture of the chamber of the heart. For example, sensing module 86 may be used to monitor for electrical signal evoked by the delivery of pacing therapy within a particular period of time after the delivery of the pacing therapy. Such an evoked electrical signal may indicate that the delivered pacing therapy effectively captured the chamber of heart 12, and the absence of such an evoked electrical signal may indicate that the delivered pacing therapy has failed to achieve capture of the chamber of heart 12. In some examples, sensing module 86 comprises one or more amplifiers or other circuitry that provides a signal to processor 80 when a signal meets amplitude or other criteria for an evoked response, and processor 80 determines whether an evoked response occurred based on the timing of the signal from sensing module 86.

Telemetry module 88 includes any suitable hardware, firmware, software or any combination thereof for communicating with another device, such as programmer 24 (FIG. 1). Under the control of processor 80, telemetry module 88 may receive downlink telemetry from and send uplink telemetry to programmer 24 with the aid of an antenna, which may be internal and/or external. Processor 80 may provide the data to be uplinked to programmer 24 and receive data downlinked from programmer 24 via telemetry module 88.

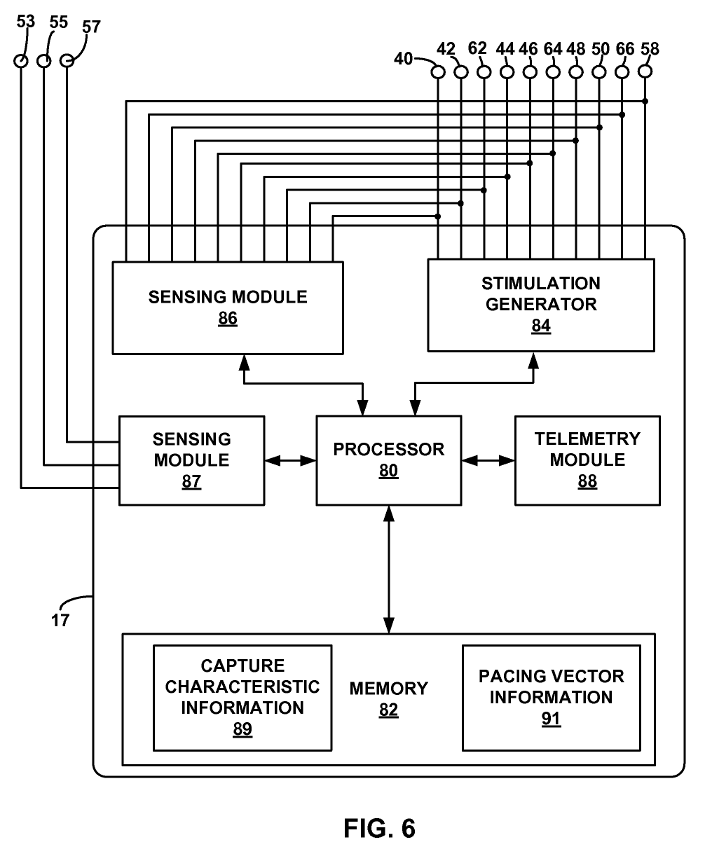

FIG. 6 is a functional block diagram of example of IMD 17 of FIG. 3, which is similar to IMD 16 except that IMD 17 may also include sensing module 87. As previously described with respect to FIG. 3, IMD 17 is coupled to lead-based sensors 53, 55, and 57, which may sense signals associated one or more physiological parameters. IMD 17 includes sensing module 87, in addition to sensing module 86, to support processing or signals sensed by sensors 53, 55, 57. In some implementations, sensing module 87 may be integrated with sensing module 86 or share some common hardware, firmware or software with sensing module 86 and/or processor 80. In some examples, sensing module 87 comprises one or more amplifiers, filters, analog-to-digital converters, or the like, to condition the signals from sensors 53, 55, 57 for receipt and processing by processor 80.

Signals generated by sensors 53, 55 and 57 may indicate mechanical contraction of heart 12 or otherwise indicate capture of the heart by a pacing stimulus. For example, one or more of sensors 53, 55 and 57 may include an accelerometer. Sensing module 87 and/or processor 80 may monitor the accelerometer signal after delivery of pacing therapy to a chamber of heart 12 via a pacing vector to determine if a contraction of heart 12 was evoked by the therapy. In this manner, processor 80 may monitor heart 12 to determine whether the delivery of pacing therapy to heart 12 via a particular pacing vector is adequately capturing the chamber of heart 12.

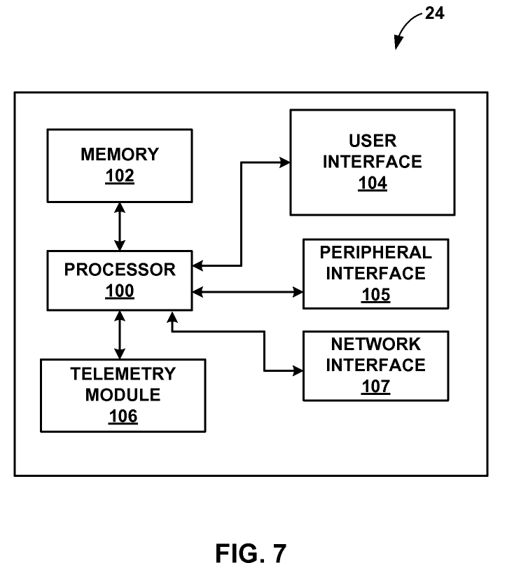

FIG. 7 is block diagram of an example configuration of programmer 24. As shown in FIG. 7, programmer 24 includes processor 100, memory 102, user interface 104, peripheral interface 105, telemetry module 106, and network interface 107. Programmer 24 may be a dedicated hardware device with dedicated software for programming of IMD 16. Alternatively, programmer 24 may be an off-the-shelf computing device running an application that enables programmer 24 to program IMD 16.

A user, such as a clinician or other caregiver, interacts with programmer 24, and more particularly processor 100, via user interface 104, which may include display to present graphical user interface to a user, and a keypad or another mechanism for receiving input from a user. Processor 100 can take the form one or more microprocessors, DSPs, ASICs, FPGAs, programmable logic circuitry, or the like, or any combination thereof. The functions attributed to processor 100 herein may be embodied as hardware, firmware, software or any combination thereof.

Memory 102 may comprise one or more memory modules or data storage devices, and may store instructions that cause processor 100 to provide the functionality ascribed to programmer 24 herein, and information used by processor 100 to provide the functionality ascribed to programmer 24 herein. Memory 102 may include any fixed or removable magnetic, optical, or electrical media, such as RAM, ROM, CD-ROM, hard or floppy magnetic disks, EEPROM, or the like. Memory 102 may also include a removable memory portion that may be used to provide memory updates or increases in memory capacities. Memory 102 may also store information that controls therapy delivery by IMD 16, such as stimulation parameter values, e.g., such as voltage or current amplitude, pulse width, frequency, blanking intervals, escape intervals, or the like.

Programmer 24 may communicate wirelessly with IMD 16, e.g., using RF communication or proximal inductive interaction. This wireless communication may be performed through the use of telemetry module 106, which may be coupled to an internal antenna or an external antenna. Telemetry module 106 may also be configured to communicate with another computing device via wireless communication techniques, or direct communication through a wired connection. Examples of local wireless communication techniques that may be employed to facilitate communication between programmer 24 and another computing device include RF communication according to the 802.11 or Bluetooth specification sets, infrared communication, e.g., according to the IrDA standard, or other standard or proprietary telemetry protocols. In this manner, other external devices may be capable of communicating with programmer 24 without needing to establish a secure wireless connection.

Programmer 24 may also include peripheral interface 105 to connect to one or more peripheral devices. For example, peripheral interface 105 may include one or more suitable peripheral interface controllers, e.g., a UBS controller or the like, that allows programmer 24 to connect with a desired peripheral device, e.g., an external memory storage medium.

Programmer 24 may also be configured to communicate with one or more network devices via network interface 107. For example, network interface 107 may include one or more suitable network interface controllers, e.g., an Ethernet port or the like, that allows programmer 24 to connect with a desired network device, e.g., a network server.

In some examples, processor 100 may perform one or more of the functions attributed to processor 80 of IMD 16 that are described herein. For example, processor 100 may receive pacing information from IMD 16 via telemetry module 106 to determine whether pacing therapy delivered to a chamber of heart 12 via a first electrode configuration inadequately captures the chamber of the heart. Processor 100 may also determine a capture characteristic for each additional pacing vector based on information communicated from IMD 16 relating to the delivery of pacing therapy to the chamber of heart 12 via each additional pacing vector. In some cases, processor 100 may automatically select a new pacing vector from the additional pacing vectors based on the capture characteristics determined for each additional pacing vector. Additionally or alternatively, user interface 104 may be configured to provide capture characteristic information determined for one or more of the each additional pacing vector to a user, such as, e.g., a clinician or patient 14. The user may review the provided capture characteristic information and interact with programmer 24 via user interface 104 to select a new pacing vector based on the provided capture characteristic information. Processor 100 may then communicate the selection to IMD 16 via telemetry module 106.

FIG. 8 is a simplified schematic diagram illustrating IMD 16 of therapy system 10. FIG. 8 illustrates various pacing vectors through which IMD 16 may deliver pacing therapy to respective chambers of heart 12. IMD 16 includes all features as described previously, including implantable leads 18, 20, and 22. The location of lead 18, 20, and 22 with respect to the chambers of heart 12 is indicated by dashed sections corresponding to RA 26, RV 28, and LV 32. However, the path followed by leads 18, 20, and 22 to IMD housing 60 with respect to the dashed sections indicated by FIG. 8 are not necessarily representative of an actual configuration of an IMD 16 implanted in the heart of a patient.

IMD 16 may utilize a variety of pacing vectors to capture a chamber of heart 12 via delivery pacing therapy. In some examples, a pacing vector is a single lead pacing vector, i.e., including electrode(s) from only one of leads 18, 20, and 22. For example, IMD 16 may generate and deliver pacing therapy to LV 32 of heart 12 via a bipolar pacing vector defined by anodic electrode 44 and cathodic electrode 46 of lead 20, indicated by arrow 71. Throughout the description of FIG. 8, the tip of an arrow indicating a pacing vector generally corresponds to the anodic electrode and the tail of the arrow generally corresponds to the cathodic electrode. Accordingly, arrow 75 represents a pacing vector defined by cathodic electrode 44 and anodic electrode 46, which is a different pacing vector than that of pacing vector 71 although both utilize electrodes 44 and 46. In each case, IMD 16 may deliver pacing therapy to LV 32 of heart 12 via pacing vector 71 or pacing vector 75.

In other examples, a bipolar pacing vector may be multi-lead pacing vector, i.e., including at least two electrodes that are provided on separate implantable leads. For example, IMD 16 may deliver pacing therapy to heart 12 via a pacing vector defined by cathodic electrode 50 of lead 22 and anodic electrode 40 of lead 18, indicated by arrow 72. Similarly, IMD 16 may deliver pacing therapy to heart 12 via a pacing vector defined by anodic electrode 50 of lead 22 and cathodic electrode 40 of lead 18, indicated by arrow 75.

In still other examples, electrode 58 of IMD housing 60 may be included as an electrode in a unipolar pacing vector, e.g., unipolar pacing vectors defined by electrode 58 and any one of electrodes 40, 42, 44, 46, 48, 50, 62, 64, and 66. For example, IMD 16 may deliver pacing therapy to heart 12 via a pacing vector defined by anodic electrode 58 of IMD housing 60 and cathodic electrode 64 of lead 20, as indicated by arrow 73. Similarly, IMD 16 may deliver pacing therapy to heart 12 via a pacing vector defined by cathodic electrode 58 of IMD housing 60 and anodic electrode 64 of lead 20, as indicated by arrow 79.

Furthermore, in some examples, the configuration of respective pacing vectors used by IMD 16 to deliver pacing therapy to a respective chamber of heart 12 is not limited by the type of electrodes used. In some cases, a pacing vector may include electrodes of the same type, e.g., ring electrodes 40 and 44. Additionally, in some cases, a pacing vector may include electrodes of different types, e.g., a pacing vector including ring electrode 44 and helix tip electrode 46, or ring electrode 44 and elongated coil electrode 64, or can electrode 58 and coil electrode 62 or 64.

In some examples, the electrode(s) used for sensing cardiac signals, e.g., those cardiac signals used by processor 80 (FIGS. 5 and 6) to determine whether the pacing therapy delivered via a pacing vector is achieving adequate capture in a chamber and/or determine a capture characteristic of a respective pacing vector, are different than the pacing vectors used by IMD 16 to deliver pacing therapy to heart 12. For example, if cardiac signals are sensed via a sensing vector including electrodes 40 and 42 of lead 18, IMD 16 may deliver pacing therapy via a pacing vector including any of electrodes 40, 42, and 62 from lead 18, or any of electrodes 44, 46, 48, 50, 58, 64, and 66 associated with other leads or with IMD housing 60. Accordingly, in some examples, delivery of pacing therapy and the sensing of cardiac signals may utilize electrodes from a single implantable lead. Alternatively, the sensing vector and pacing vector may include the same electrodes on the same lead or leads.

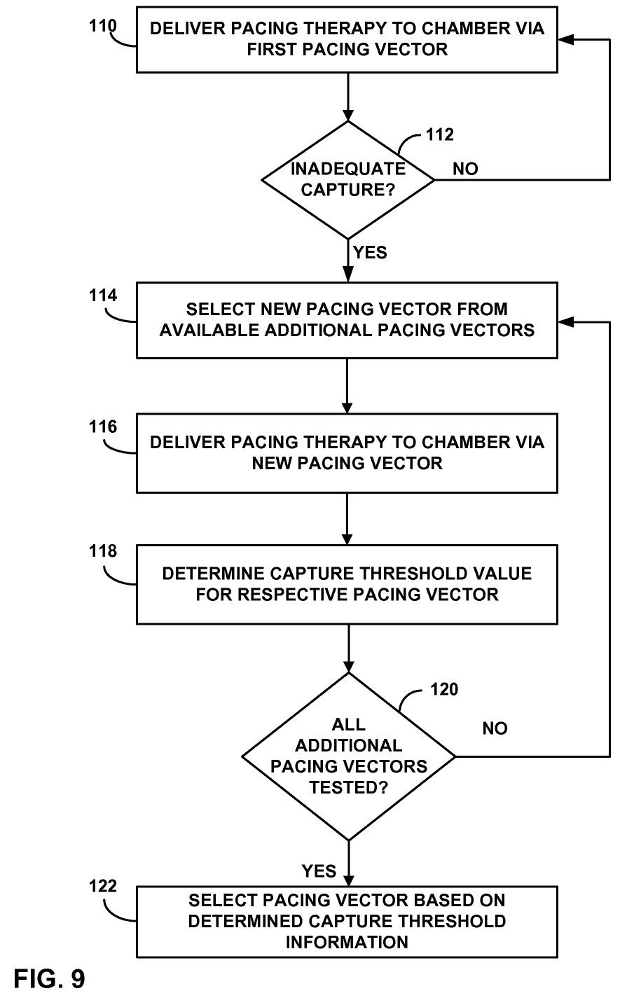

FIG. 9 is a flow diagram illustrating an example technique according to one aspect of this disclosure. Such an example technique may be useful for managing the delivery of pacing therapy from therapy system 10 to heart of patient 14. For the purposes of illustration, the example technique will be described with respect to therapy system 10 of this disclosure. However, such a technique is not limited to systems with such configurations but instead may be utilized in any system for which the technique may be suitably applied. Furthermore, although the example technique of FIG. 9 is described with respect to delivery of pacing therapy to the LV of heart 12, examples are not limited only the LV of the heart but may be appropriate for pacing of any chamber of heart 12.

As indicated in FIG. 9, IMD 16 generates and delivers pacing therapy to a chamber of heart 12 via a first pacing vector (110). For example, IMD 16 may generate and deliver pacing therapy to LV 32 via pacing vector defined by anodic electrode 46 and cathodic electrode 44 (pacing vector 75 in FIG. 8). As previously explained, IMD 16 may deliver the pacing therapy delivered treat a cardiac rhythm disorder of heart 12 of patient 14.

After delivering the pacing therapy via pacing vector 75, processor 80 of IMD 16 determines whether the pacing therapy inadequately captures LV 32 of heart 12 (112). As previously described, to evaluate the adequacy of capture, processor 80 may analyze one or more cardiac signals monitored by sensing module 86 via one or more sensing vectors or sensors to determine if the pacing therapy delivered via pacing vector 75 has evoked a response indicative of capture of LV 32.

In some examples, processor 80 may determine whether the pacing therapy delivered via the pacing vector 75 inadequately capture LV 32 based on evaluation of the delivery of a single pacing pulse or a series of pacing pulse configured to evoked a single contraction within LV 32. For example, IMD 16 may generate and delivery a single pacing pulse to LV 32 via pacing vector 75. Sensing module 86 may subsequently monitor the electrical signals of heart 12 to determine whether an evoked signal indicative of capture of LV 32 is sensed. If such an evoked signal is sensed, processor 80 may determine that delivery of the pacing stimulation via pacing vector 75 adequately captures LV 32. Conversely, if an evoked signal is not sensed via sensing module 86 or an evoked signal that is not indicative of LV 32 capture, then processor 80 may determine that delivery of the pacing therapy via pacing vector 75 inadequately captures LV 32.

Alternatively, processor 80 may determine whether the pacing therapy delivered via the pacing vector 75 inadequately capture LV 32 based on evaluation of the delivery of pacing therapy intended to provide multiple contractions of LV 32 over of longer period of time. For example, in a case in which the pacing therapy includes a single pulse that is configured to capture LV 32, sensing module 86 may monitor the electrical activity of heart 12 during a period of time in which IMD 16 delivers a plurality of pacing pulses to LV 32 via pacing vector 75, which are intended to evoke a plurality of contractions in LV 32. Processor 80 may then analyze the electrical activity and/or sensor signals to determine what percentage of pacing pulses captured the LV and/or what percentage of pacing pulses did not capture the LV.

In such cases, if the percentage of pacing pulses that evoked an electrical signal indicative of LV capture is greater than or equal to a threshold value, then processor 80 determines that delivery of pacing therapy to LV 32 via pacing vector 75 adequately captures LV 32. Conversely, if the percentage of pacing pulses that evoked an electrical signal indicative of LV capture is less than a threshold value, processor 80 determines that delivery of pacing therapy to LV 32 via pacing vector 75 inadequately captures LV 32. The threshold value used by processor 80 to evaluate whether the delivery of pacing therapy via pacing vector 75 adequately captures LV 32 may be any value for appropriate for evaluating the adequacy of the pacing therapy being delivered via pacing vector 75.

In still another example, processor 80 may determine that delivery of pacing therapy to LV 32 via pacing vector 75 inadequately captures LV 32 based on the intensity of the pacing therapy required to capture LV 32. For example, the stimulation parameters of the pacing therapy are such that they may be considered inefficient with respect to power consumption, or too near a maximum output of IMD 16 to reliably capture heart 12, processor 80 may determine that the delivery of pacing therapy to LV 32 via pacing vector 75 inadequately captures LV 32. An example of a stimulation parameter being too near a maximum value is an amplitude required for capture being less than a safety margin away from a maximum amplitude available from IMD 16.

In any case, if processor 80 determines that the pacing therapy delivered via pacing vector 75 adequately captures LV 32, IMD 16 continues to deliver pacing therapy to LV 32 via pacing vector 75. However, if processor 80 determines that the pacing therapy delivered via pacing vector 75 inadequately capture LV 32, processor 80 initiates a process that may be used to select a new pacing vector for delivering pacing therapy to the same chamber as pacing vector 75, i.e., LV 32.

As indicated in FIG. 9, processor 80 selects a new pacing vector other than that of pacing vector 75 from a plurality of the additional pacing vectors available to deliver pacing therapy to LV 32 (114). For example, processor 80 may access information stored in pacing vector data 91 portion of memory 82 that defines a list of additional pacing vectors corresponding to LV 32 pacing of heart 12. In some examples, the pacing vector list defines all the additional pacing vectors available for delivery of pacing stimulation to LV 32. Alternatively, the pacing vector list may be limited to additional pacing vectors that have been selected, e.g., by an clinician, as appropriate for testing in the event that delivery of pacing therapy via vector 75 is determined to be inadequate.

For purposes of illustration, FIG. 9 will be described with respect to a scenario wherein the plurality of additional pacing vectors defined within pacing vector data 91 portion of memory 82 include a total of three pacing vectors; pacing vectors 71, pacing vector 81, i.e., the pacing vector defined by anodic electrode 64 and cathodic electrode 44, and pacing vector 83, i.e., the pacing vector defined by anodic electrode 46 and cathodic electrode 64 (all illustrated in FIG. 8). However, any number of additional pacing vectors are contemplated.

After processor 80 selects the new pacing vector, e.g., pacing vector 71 (114), IMD 16 delivers pacing therapy to LV 32 via pacing vector 71 (116). Processor 80 determines a capture threshold value during the delivery of the pacing therapy to LV 32 via pacing vector 71. As indicated by FIG. 9, this process may be repeated with pacing vector 81 and then pacing vector 83 to determine a capture threshold value for each respective pacing vector. Processor 80 may store the capture threshold value for each respective pacing vector in capture characteristic information 89 portion of memory 82.

Processor 80 may determine the capture threshold value for each pacing vector using any suitable technique known in the art. In some examples, the capture threshold value refers to a voltage amplitude threshold. In other examples, the capture threshold value refers to a current amplitude threshold or pulse width threshold.

To determine that voltage amplitude threshold for a respective pacing vector, IMD 16 may deliver pacing therapy including an electrical signal with a relatively high voltage amplitude via the respective pacing vector. Processor 80 may then determine whether the pacing therapy achieved LV capture. If so, processor 80 may then deliver pacing therapy including an electrical signal with a decreased voltage amplitude via the same pacing vector. Processor 80 may again determine whether the pacing therapy achieved LV capture. This process is repeated while iteratively decreasing the voltage amplitude until processor 80 detects that pacing therapy delivered via the pacing vector at a specific voltage amplitude did not achieve LV capture. In such cases, processor 80 may define the voltage amplitude value of the pacing therapy just prior to the therapy that did not achieve LV capture as the capture threshold value. In some examples, the capture threshold value may be defined as the voltage amplitude value of the pacing therapy just prior to the therapy that did not achieve LV capture, plus a nominal amplitude value added to provide a measure of insurance, i.e., a safety margin, as the capture threshold value.

Alternatively or additionally, a capture characteristic other than that of capture threshold may be determined for each additional pacing vector. For example, while FIG. 9 is described with respect pacing threshold values, examples of the present disclosure may include any suitable capture characteristic. In general, a capture characteristic may be any information that allows for each of the additional pacing vectors to be evaluated relative to one another with respect to LV pacing on a level other than that of arbitrarily. In a relatively simple example, the capture characteristic for each pacing vector may simply be an indicated of whether delivery of the pacing therapy to LV 32 via the respective pacing vector adequately captured LV 32. Processor 80 may determine such information the same or similar to that described above with respect to the adequacy of LV capture with respect to pacing vector 75 (112).

In other examples, the capture characteristic determined for each pacing vector may include pacing impendence, sensed cardiac amplitude, timing between the local sensing of evoked cardiac signal and sensing of evoked cardiac signal in other locations of heart 12, and/or phrenic nerve capture, e.g., as identified via one or more accessory sensors, such as an accelerometer.

In some examples, the delivery of pacing therapy for purposed of determining the capture characteristic of each additional pacing vector is with delivery of pacing therapy via pacing vector 75, i.e., the primary or current pacing vector that processor 80 determined had inadequate capture of LV 32. For example, this may be the case in situations in which the delivery of pacing therapy via pacing vector 75 still may provide for LV capture, albeit at less than ideal percentage and/or at relatively undesirable stimulation parameter levels, while the ability of other vectors to capture is unknown. In this manner, LV 32 may not be subject to extended periods without a suitable level of pacing in the event that the additional pacing vector are not successful in capturing LV 32. In some examples, backup pacing delivered via the primary pacing vector (75 in this example) may occur at or near a maximum amplitude or pulse width in order to make capture with the primary pacing vector more likely.

Once a capture characteristic has been determined for all of the additional pacing vectors, a new pacing vector may be selected for regular pacing stimulation that replaced pacing vector 75 based on the capture threshold information determined for each additional pacing vector. Because of the differences between the configurations of pacing vectors 71, 81, and 83, the capture threshold values determined for each pacing vector may be different from one another. On this principal, a new pacing vector may be selected from the plurality of additional pacing to replace pacing vector 75 based at least in part of the capture threshold values determined for each pacing vector.

In some cases, processor 80 may automatically select a new pacing vector to replace pacing vector 75. For example, processor 80 may automatically select the additional pacing vector that corresponds to the lowest capture threshold value. In this manner, processor 80 may select what may be considered the most power efficient pacing vector, assuming a direct correlating with voltage amplitude. As another example, in cases in which the capture characteristic is simply an indication of whether LV capture was achieved by delivery of pacing therapy via each additional pacing vector, processor 80 may simply select the first pacing vector from the list of additional pacing vectors that achieved LV capture.

Alternatively or additionally, the capture threshold information stored in capture characteristic information 89 portion of memory 82 may be transferred from IMD 16 to programmer 24 via telemetry modules 88 and 106. In some examples, processor 100 of programmer 24 may evaluate the additional pacing vectors relative to one another based on the capture threshold values determined for each pacing vector and then automatically select a new pacing vector to replace pacing vector 75, in a manner similar to that described with respect to processor 80.

In another example, the capture threshold value for one or more of the additional pacing vectors may be provided to a user, e.g., a clinician or patient 14. For example, programmer 24 may display one or more or the additional pacing vectors and it's correspond capture threshold to a user via user interface 104. The user, e.g., a clinician or patient 14, may evaluate the information and then select the new pacing vector from pacing vectors 71, 81, and 83 to replace pacing vector 75. In some cases, the user may manually evaluate the capture threshold values and then input a desired pacing vector by selecting the pacing vector for a list of the additional pacing vectors. In other cases, the user may be simply prompted to authorize a new pacing vector that has been pre-selected by processor 80 or 100.

Once a pacing vector has been selected, programmer 24 may communicate the selection to IMD 16. IMD 16 may receive the signal from programmer 24 and then processor 80 may reconfigure the therapy delivery parameters based on the pacing vector indicated in the signal received from programmer 24. Processor 80 controls signal generator 84 to deliver pacing stimulation via the newly selected pacing vector.

The techniques described in this disclosure, including those attributed to IMD 16, programmer 24, or various constituent components, may be implemented, at least in part, in hardware, software, firmware or any combination thereof. For example, various aspects of the techniques may be implemented within one or more processors, including one or more microprocessors, DSPs, ASICs, FPGAs, or any other equivalent integrated or discrete logic circuitry, as well as any combinations of such components, embodied in programmers, such as physician or patient programmers, stimulators, image processing devices or other devices. The term “processor” or “processing circuitry” may generally refer to any of the foregoing logic circuitry, alone or in combination with other logic circuitry, or any other equivalent circuitry.

Such hardware, software, firmware may be implemented within the same device or within separate devices to support the various operations and functions described in this disclosure. While the techniques described herein are primarily described as being performed by processor 80 of ICD 16 and/or processor 100 of programmer 24, any one or more parts of the techniques described herein may be implemented by a processor of one of the IMD 16, programmer 24 or another computing device, alone or in combination with IMD 16 or programmer 24.

In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

When implemented in software, the functionality ascribed to the systems, devices and techniques described in this disclosure may be embodied as instructions on a computer-readable medium such as RAM, ROM, NVRAM, EEPROM, FLASH memory, magnetic data storage media, optical data storage media, or the like. The instructions may be executed to support one or more aspects of the functionality described in this disclosure.

Various embodiments of the invention have been described. These and other embodiments are within the scope of the following claims.

1. A method comprising:
delivering cardiac pacing therapy from a medical device to a chamber of a heart via a first electrode configuration;
determining that the delivery of cardiac pacing therapy via the first electrode configuration inadequately captures the chamber;
delivering cardiac pacing therapy to the chamber of the heart via a plurality of additional electrode configurations in response to the determination that the delivery of cardiac pacing therapy via the first electrode configuration inadequately captures the chamber; and
determining a capture characteristic for each of the additional electrode configurations based on the delivery of cardiac pacing therapy to the chamber of the heart via the plurality of additional electrode configurations.

2. The method of claim 1, wherein determining the capture characteristic for each of the additional electrode comprises determining a capture threshold for each of the additional electrode configurations.

3. The method of claim 1, further comprising providing the capture characteristics to a user.

4. The method of claim 3, further comprising:
receiving an indication from a user indicating one of the additional electrode configurations; and
selecting one of the additional electrode configurations based at least in part on the indication.

5. The method of claim 1, further comprising selecting one of the additional electrode configurations for delivery of cardiac pacing therapy from the medical device to the chamber of the heart based on the determined capture characteristics.

6. The method of claim 5, wherein a processor of the medical device automatically selects one of the additional electrode configurations based on the determined capture characteristics.

7. The method of claim 1, wherein the chamber of the heart comprises one of a left ventricle or right ventricle.

8. The method of claim 1, wherein determining that the delivery of cardiac pacing therapy via the first electrode configuration inadequately captures the chamber comprises:
monitoring one or more cardiac signals subsequent the delivery of the cardiac pacing therapy to the chamber of the heart via the first electrode configuration; and
determining that the one or more cardiac signals are not indicative of capture of the chamber.

9. The method of claim 1, wherein determining that the delivery of cardiac pacing therapy via the first electrode configuration inadequately captures the chamber comprises:
evaluating one or more stimulation parameter values of the cardiac pacing therapy delivered to the chamber of the heart via the first electrode combination; and
determining that the delivery of cardiac pacing therapy via the first electrode configuration inadequately captures the chamber based on the evaluation of the one or more stimulation parameter values of the cardiac pacing therapy.

10. A medical device system comprising:
a stimulation generator configured to deliver cardiac pacing therapy to a chamber of a heart via a first electrode configuration; and
a processor configured to determine that the delivery of cardiac pacing therapy via the first electrode configuration inadequately captures the chamber,
wherein the stimulation generator is configured to deliver cardiac pacing therapy to the chamber of the heart via a plurality of additional electrode configurations in response to the determination that the delivery of cardiac pacing therapy via the first electrode configuration inadequately captures the chamber,
wherein the processor is configured to determine a capture characteristic for each of the additional electrode vector configurations based on the delivery of cardiac pacing therapy to the chamber of the heart via a plurality of additional electrode configurations.

11. The medical device system of claim 10, wherein determining the capture characteristic for each of the additional electrode comprises determining a capture threshold for each of the additional electrode configurations.

12. The medical device system of claim 10, further comprising a user interface configured to provide the capture characteristics to a user.

13. The medical device system of claim 12, wherein the processor is configured to receive an indication from a user indicating one of the additional electrode configurations and select one of the additional electrode configurations based at least in part on the indication.

14. The medical device system of claim 10, wherein the processor is configured to select one of the additional electrode configurations for delivery of cardiac pacing therapy from the medical device to the chamber of the heart based on the determined capture characteristics.

15. The medical device system of claim 14, wherein the processor is configured to automatically select one of the additional electrode configurations based on the determined capture characteristics.

16. The medical device system of claim 10, wherein the chamber of the heart comprises one of a left ventricle or right ventricle.

17. The medical device system of claim 10, further comprising:
a sensing module configured to monitor one or more cardiac signals subsequent the delivery of the cardiac pacing therapy to the chamber of the heart via the first electrode configuration,
wherein the processor is configured to determine that the delivery of cardiac pacing therapy via the first electrode configuration inadequately captures the chamber when the one or more cardiac signals monitored via the sensing module subsequent the delivery of the cardiac pacing therapy to the chamber of the heart via the first electrode configuration are not indicative of capture of the chamber.

18. The medical device system of claim 10, wherein the processor is configured to evaluate one or more stimulation parameter values of the cardiac pacing therapy delivered to the chamber of the heart via the first electrode combination,
wherein the processor is configured to determine that the delivery of cardiac pacing therapy via the first electrode configuration inadequately captures the chamber based at least in part on the evaluation of the one or more stimulation parameter values of the cardiac pacing therapy.

19. A medical device system comprising:
means for delivering cardiac pacing therapy from a medical device to a chamber of a heart via a first electrode configuration;
means for determining that the delivery of cardiac pacing therapy via the first electrode configuration inadequately captures the chamber;
means for delivering cardiac pacing therapy to the chamber of the heart via a plurality of additional electrode configurations in response to the determination that the delivery of cardiac pacing therapy via the first electrode configuration inadequately captures the chamber; and
means for determining a capture characteristic for each of the additional electrode configurations based on the delivery of cardiac pacing therapy to the chamber of the heart via a plurality of other electrode configurations.

20. The medical device system of claim 19, further comprising means for providing the capture characteristics to a user.
